(19) United States
(12) Patent Application Publication
(10) Pub. No.: US 2023/0138666 A1
(43) Pub. Date: May 4, 2023

(54) INTRAOPERATIVE 2D/3D IMAGING PLATFORM

(71) Applicant: Memorial Sloan Kettering Cancer Center, New York, NY (US)

(72) Inventor: Bryan C. HUSTA, New York, NY (US)

(73) Assignee: Memorial Sloan Kettering Cancer Center, New York, NY (US)

(21) Appl. No.: 17/794,340

(22) PCT Filed: Jan. 25, 2021
(86) PCT No.: PCT/US21/14853

§ 371 Date: Jul. 21, 2022

Related U.S. Application Data
(60) Provisional application No. 62/965,264, filed on Jan. 24, 2020.

(51) Int. Cl. A61B 6/12; A61B 1/267; A61B 1/00; A61B 6/00

(52) U.S. Cl. CPC A61B 6/12; A61B 1/2676; A61B 1/00147; A61B 6/5235; A61B 6/463; A61B 6/466; A61B 2090/3762

(57) ABSTRACT

The present disclosure is directed to systems and methods for intraoperative medical imaging A computing system may access, from a database, a first tomogram derived from scanning a volume within a subject prior to an invasive procedure. The first tomogram may identify a target within the volume of the subject. The computing system may acquire data via an endoscopic device within the subject at a time instance during the invasive procedure. The computing system may provide, for display, in the first tomogram of the subject, a first relative location of a distal end of the endoscopic device and the target based on the data. The computing system may receive a second tomogram of the volume at the time instance. The computing system may register the second tomogram with the first tomogram to determine a second relative location of the distal end and the target.

### CROSS REFERENCE TO RELATED APPLICATIONS

The present application claims priority to U.S. Provisional Patent Application No. 62/965,264, titled “Inoperative 2D/3D Imaging Platform for Performing Lung Biopsies,” filed Jan. 24, 2020, which is incorporated herein by reference in its entirety.

### BACKGROUND

Medical imaging may be used to acquire visual representations of an interior of a body beneath the outer tissue of the patient. The visual representations may be two-dimensional or three-dimensional, and may be used for diagnosis and treatment of the patient.

### SUMMARY

Diagnostic bronchoscopy procedures make use of guided bronchoscopy in order to reach target sites in the periphery of the lung. This can be accomplished by use of radial ultrasound bronchoscope and electromagnetic navigation bronchoscopy. The development of robotic bronchoscopy has provided more precision for peripheral lung procedures. Navigation tools such as electromagnetic navigation bronchoscopy as well as robotic bronchoscopy make use of a pre-procedure CT scan on full inhalation of the chest and use it as a road map for guiding the bronchoscope to an intended target site. Since real-time data is not used for guidance in the periphery of the lung the guidance used in robotic bronchoscopy and electromagnetic navigation bronchoscopy are all virtually calculated. All of these tools may be aided by confirmation either with intraoperative fluoroscopic C-arm or Cone-beam CT scan in order to be certain that the tools used for biopsies are in the intended location within the lung.

A circumferential imaging system can be an intraoperative 2D/3D imaging system designed for use in a variety of procedures including spine, cranial, and orthopedics. The circumferential imaging system is a mobile X-ray system designed for 2D fluoroscopic and 3D imaging for adult and pediatric patients and is intended to be used where a physician benefits from 2D and 3D information of anatomic structures and objects with high x-ray attenuation such as bony anatomy and metallic objects. The circumferential imaging system can be used for confirming the position of endoscopic tools in the lung. Furthermore, an interface can be provided whereby target sites, anatomical structures and various tools deployed in the periphery of the lung using robotic bronchoscopy can be identified with the real-time 3-D scan provided by the circumferential imaging system. After the position of the tools, anatomical structures and the target site is identified on the 3-D scan, adjustments can be made to the position of the tools in the lung to the desired position using the robotic bronchoscope. By merging the virtual three-dimensional position data from the robotic bronchoscope and the real-time position of tools, anatomic structures and the target sites all identified by the three-dimensional scan a more accurate local representation of the position of these objects can be constructed to further guide procedures in the periphery of the lung with more accuracy.

By combining the robotic bronchoscopy navigation and the intraoperative 3D images obtained by the circumferential imaging system, there is a potential for added accuracy and increased diagnostic yield for biopsies performed in the periphery of the lung. Additionally, with the ability to identify anatomic structures that should be avoided such as prominent blood vessels and the distance to the outer lining of the lung there is a potential for increased safety profile for these procedures. Finally, as local therapeutic procedures are being developed in the periphery of the lung, more accurate confirmation of the real-time position of these tools is imperative in order to ensure accurate delivery of energy therapies and to improve safety by avoiding proximity to critical structures.

Aspects of the present disclosure are directed to systems, methods, devices, and non-transitory computer-readable media for intraoperative medical imaging. A computing system having one or more processors coupled with memory may access, from a database, a first tomogram derived from scanning a volume within a subject prior to an invasive procedure. The first tomogram may identify a target within the volume of the subject. The computing system may acquire data via an endoscopic device at least partially disposed within the subject at a time instance during the invasive procedure. The computing system may provide, for display, in the first tomogram of the subject, a first relative location of a distal end of the endoscopic device and the target based on the data. The computing system may receive, using a tomograph, a second tomogram of the volume within the subject at the time instance during the invasive procedure. The second tomogram may include the distal end of the endoscopic device. The computing system may register the second tomogram received from the tomograph during the invasive procedure with the first tomogram obtained prior to the invasive procedure to determine a second relative location of the distal end of the endoscopic device and the target within the subject. The computing system may provide, for display, the second relative location of the distal end and the target within the subject during the invasive procedure.

In some embodiments, the computing system may receive, using the tomograph, a third tomogram of the volume within the subject at a second time instance during the invasive procedure after the time instance. The third tomogram may include the distal end of the endoscope moved subsequent to provision of the second relative location. In some embodiments, the computing system may register the third tomogram received from the tomograph at the second time instance with the first tomogram received prior to the invasive procedure to determine a third relative location of the distal end of the endoscopic device and the target within the subject. In some embodiments, the computing system may provide, for display, the third relative location of the distal end and the target within the subject.

In some embodiments, the computing system may provide a graphical user interface for display of one or more of: the first tomogram, the first relative location or the second relative location of the distal end in the first tomogram, a first location of the target in the first tomogram, the second tomogram, the second relative location of the distal end in the second tomogram, and a second location of the target in the second tomogram.

In some embodiments, the computing system may identify a three-dimensional representative model derived from scanning the volume within the subject prior to the invasive procedure. The three-dimensional representative model may identify an organ within the subject, one or more cavities within the organ, and the target.

In some embodiments, the computing system may acquire, via the endoscopic device, the data comprising at least one of image data acquired via the distal end of the endoscopic device and operational data identifying a translation of the endoscope through the subject. In some embodiments, the computing system may receive, using the tomograph, the second tomogram in at least one of a two-dimensional space or a three-dimensional space, the second tomogram in an imaging modality different from an imaging modality of the first tomogram.

In some embodiments, the computing system may register the second tomogram with the first tomogram to determine a displacement between the distal end of the endoscopic device and the target within the subject. In some embodiments, the computing system may register the second tomogram with the first tomogram to determine a displacement between the target in the first tomogram and the target in the second tomogram within the subject.

In some embodiments, the computing system may register the second tomogram with the first tomogram to determine a difference in size between the target in the first tomogram and the target in the second tomogram within the subject. In some embodiments, the invasive procedure may include a bronchoscopy, the distal end of the endoscopic device may be inserted through a tract in a lung of the subject, and the volume of the subject scanned may at least partially include the lung.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 is a block diagram of a system for intraoperative medical imaging using an intraoperative 2D/3D imaging platform in accordance with an illustrative embodiment;

FIG. 2 is an axonometric view of the system for intraoperative medical imaging in accordance with an illustrative embodiment;

FIG. 3 is a cross-sectional view of the system for intraoperative medical imaging in accordance with an illustrative embodiment;

FIG. 4A is a block diagram of an endoscope imaging operation for the system for intraoperative medical imaging in accordance with an illustrative embodiment;

FIG. 4B is a block diagram of a tomogram acquisition operation for the system for intraoperative medical imaging in accordance with an illustrative embodiment;

FIG. 4C is a block diagram of an image registration operation for the system for intraoperative medical imaging in accordance with an illustrative embodiment;

FIGS. 5A-10C are screenshots of a graphical user interface and biomedical images provided by the system for intraoperative medical imaging; and

FIG. 11 is a flow diagram of a method intraoperative medical imaging using an intraoperative 2D/3D imaging platform in accordance with an illustrative embodiment; and

FIG. 12 is depicts a block diagram of a server system and a client computer system in accordance with an illustrative embodiment.

### DETAILED DESCRIPTION

Following below are more detailed descriptions of various concepts related to, and embodiments of, systems and methods for intraoperative medical imaging. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes systems and methods of intraoperative medical imaging.

Section B describes a network environment and computing environment which may be useful for practicing various embodiments described herein.

### A. Systems and Methods of Intraoperative Medical Imaging

One approach to intraoperative medical imaging may rely on non-real-time scan of a subject, such as computed tomography (CT) scan acquired prior to the examination or surgical procedure on the subject. Due to inhalation and exhalation, the lung, however, may undergo significant movements or transformations during the course of the examination or procedure. Because of these movements, the non-real-time scan of the subject may not be dependable.

In accounting for some of these drawbacks, a robotic endoscope device may be used, and the navigation path for the endoscope through the lung may be calculated using the non-real time data. But this approach may not account for all the movements and transformations of the lung from inhalation and exhalation, and thus may still not be reliable for performance of procedures on the lung of the subject. To address these technical challenges, a circumferential imaging device that can provide real-time data may be used to confirm the location of the endoscope within the lung and to perform the guided bronchoscopy. In addition, an interface may be provided to identify target sites, anatomical structures and various tools deployed in the periphery of the lung using the scan data from the imaging device. Data from the robotic endoscope device may be combined with the real-time data to locate the endoscope device within the lung of the subject.

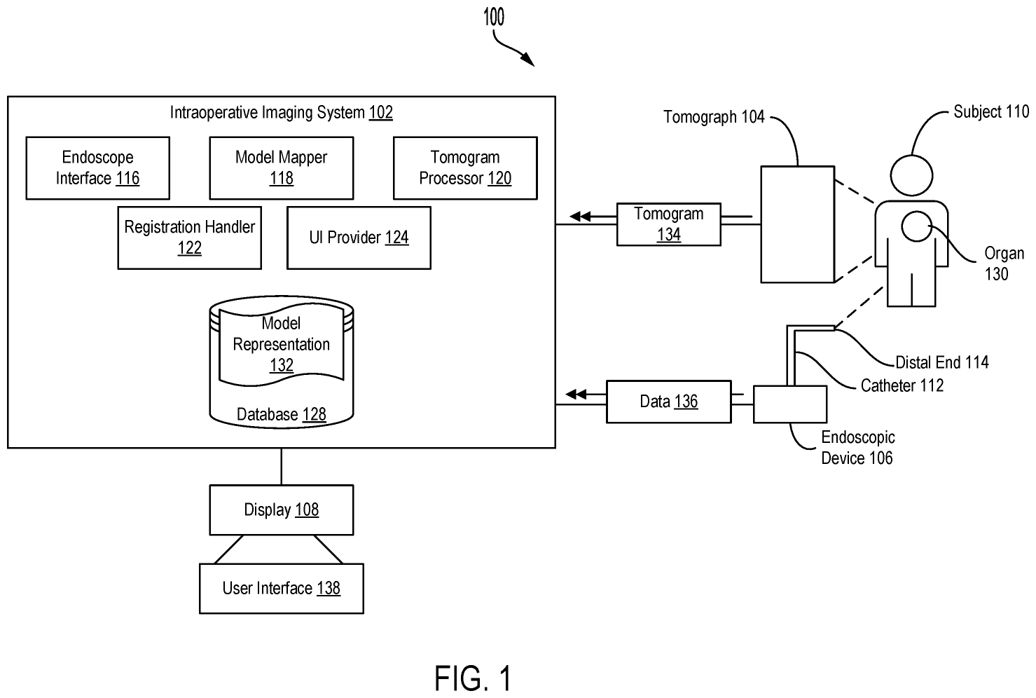

Referring now to FIG. 1, depicted is a block diagram of a system 100 for intraoperative medical imaging. In overview, the system 100 may include at least one intraoperative imaging system 102 (sometimes referred herein generally as a computing system), at least one tomograph 104, at least one endoscopic device 106, and at least one display 108, among others. The tomograph 104 and the endoscopic device 106 may be used to probe at least one organ 130 in a subject 110. The endoscopic device 106 may include at least one catheter 112 and at least one distal end 114 to be inserted into the subject 110 to examine or perform an operation on the organ 130 within the subject 130. The intraoperative imaging system 102 may include at least one endoscope interface 116, at least one model mapper 118, at least one tomogram processor 120, at least one registration handler 122, at least one user interface (UI) 124, at least one database 126, among others. The database 126 may store and maintain at least one model representation 128. The tomograph 104 may generate and provide at least one tomogram 134 to the intraoperative imaging system 102. The endoscopic device 106 may provide data 136 to the intraoperative imaging system 102. The display 108 may present at least one user interface 138 provided by the intraoperative imaging system 102. Each component described in system 100 (e.g., the intraoperative imaging system 102, the tomograph 104, and the display 108) may be implemented using one or more components of system 1200 detailed herein in Section B.

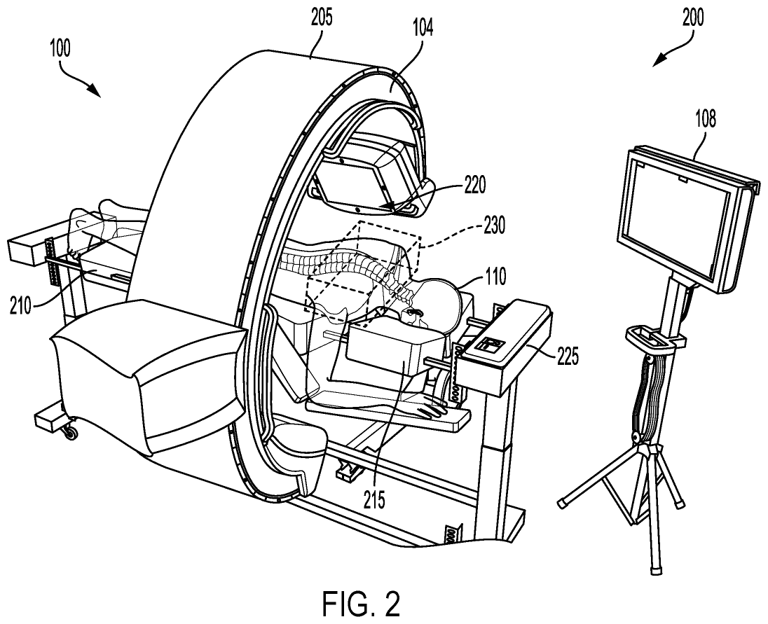

Referring now to FIG. 2, depicted is an axonometric view 200 of the system 100 for intraoperative medical imaging. As seen in the axonometric view 200, the system 100 may further include an apparatus 205 to hold, secure, or otherwise include the tomograph 104. The tomograph 104 may be a circumferential imaging device, such as a C-arm fluoroscopic imaging device as depicted. The system 100 may also include a longitudinal support 210 (e.g., a bed) and a head support 215 to hold or support the subject 110 relative to the apparatus 205 (e.g., with the subject 110 laying supine as depicted). Both the longitudinal support 210 and the head support 215 may be part of single support structure for the subject 110, and may be free of metallic components to allow for biomedical imaging of the subject 110 (e.g., x-ray penetration). The apparatus 205 may define or include a window 220 through which the subject 110 may pass to be scanned by the tomograph 104. The system 200 may also include at least one control 225 to set, adjust, or otherwise change the positioning of the longitudinal support 210 and the head support 215.

With the subject 110 situated within the apparatus 200 through window 220, the tomograph 104 may acquire the tomogram 134 of at least a portion of the subject 110. The portion of the subject 110 for which the tomogram 134 is acquired may correspond to a scanning volume 230. The portion may include at least a subset of a lung of the subject 110 and the endoscopic device 106 inserted into the lung of the subject 110. The scanning volume 230 may be defined relative to the window 220 defined by the apparatus 205 holding the tomograph 104. The tomogram 134 acquired by the tomograph 104 may be in two-dimensional or three-dimensional, or both. For example, the tomograph 104 may acquire the tomogram 134 of the scanning volume 230 of the subject 110 in layers of two-dimensional images to form a three-dimensional image. The tomogram 134 may be acquired in any number of modalities, such as an X-ray (for fluoroscopy), magnetic resonance imaging (MM), ultrasound, and positron emission tomography (PET), among others. Upon acquisition, the tomograph 104 may provide, send, or transmit the tomogram 134 to the intraoperative imaging system 102. In some embodiments, the generation and transmission of the tomogram 134 may be in real-time or near-real time (e.g., within seconds or minutes of scanning). In this manner, the subject 110 may be operated using tomogram 134s acquired of the patient in real-time.

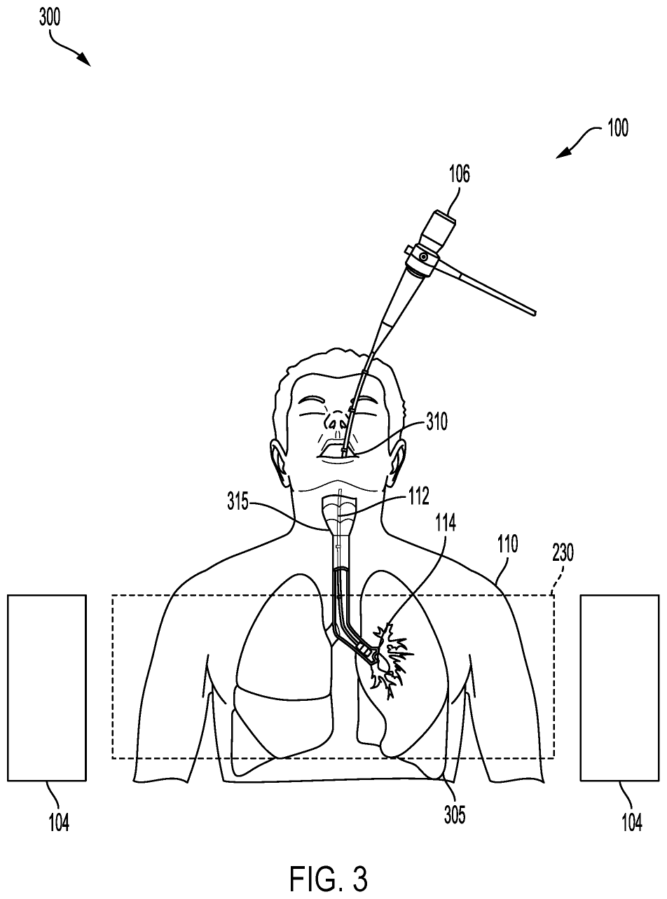

Referring now to FIG. 3, depicted is a cross-sectional view 300 of the system 100 for intraoperative medical imaging during an invasive procedure on the subject 110. The invasive procedure may involve an insertion of a tool (e.g., the endoscopic device 106 as depicted or a surgical implement) to contact the organ 130 of the subject 110. The invasive procedure may include a diagnosis or a surgical operation (e.g., a bronchoscopy), among others. As seen in the cross-sectional view 300, the scanning volume 230 may include at least one lung 305 of the subject 110. The distal end 114 and the catheter 112 of the endoscopic device 106 may be inserted into an orifice 310 (e.g., the mouth as depicted) through a respiratory tract 315 of the subject 110 to enter the lung 305. With insertion, the endoscopic device 106 may acquire data from within the lung 305 of the subject 110. The data may include, for example, an image (e.g., a visual image acquired via camera on the distal end 114 of the catheter 112) from within the lung 305, among others. Upon acquisition, the endoscopic device 106 may provide, send, or transmit the sensory data to the intraoperative imaging system 102. In some embodiments, the generation and transmission of the sensory data may be in real-time or near-real time (e.g., within seconds or minutes of scanning). While depicted as a lung in the example, the organ 130 may include the brain, heart, liver, gallbladder, kidneys, digestive tract, pancreas, and other innards of the subject 110.

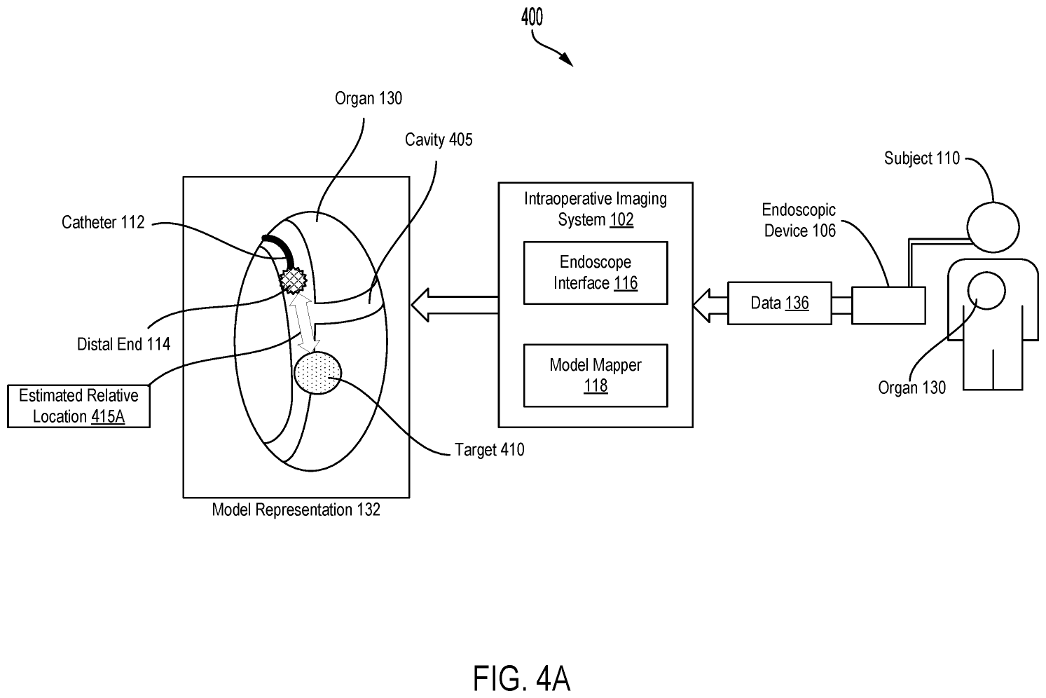

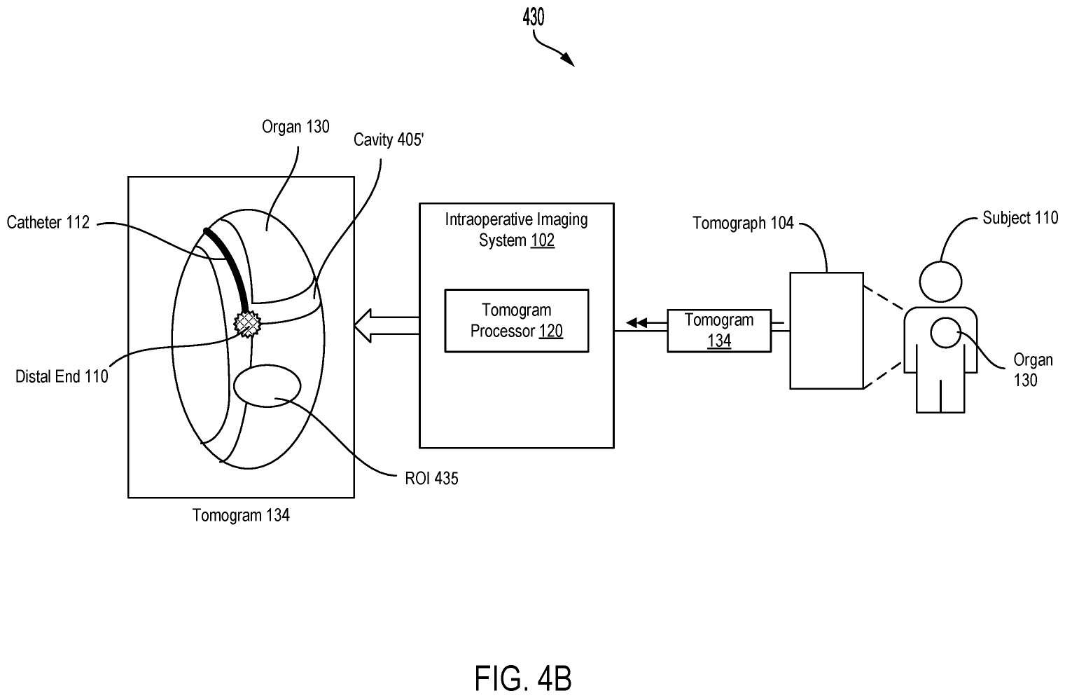

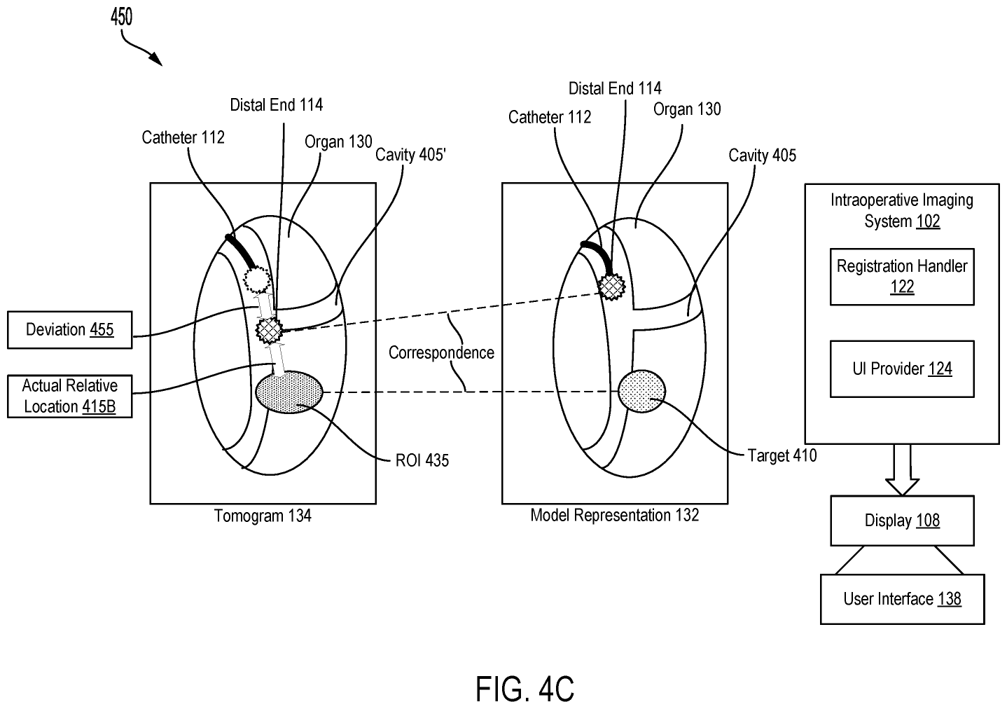

Referring now to FIG. 4A, depicted is a block diagram of an endoscope imaging operation 400 for the system 100 for intraoperative medical imaging. As depicted, the endoscope interface 116 executing on the intraoperative imaging system 102 may retrieve, identify, or receive the data 136 acquired via the endoscopic device 106. The receipt of the data 136 may be during a time instance of the invasive procedure. For example, the distal end 114 and the catheter 112 of the endoscopic device 106 may have been inserted within the subject 110 to perform a biopsy or gather measurements for diagnosis on the organ 130. The data 136 may be received by the endoscope interface 116 upon acquisition (e.g., in near real-time) by the endoscopic device 106 as the distal end 114 and the catheter 112 are moved through the subject 110. The time instance may correspond to or substantially correspond to (e.g., less than 1 minute) a time of acquisition by the endoscopic device 106. In some embodiments, the data acquired via the endoscopic device 106 may include image data from the distal end 114 (e.g., using a camera). The image data may be, for example, a capture of a visible spectrum from within a tract of the lung in the subject 110 or a sonogram from within the subject 110. In some embodiments, the data acquired via the endoscopic device 106 may include operational data of the endoscope device 106. The operational data may include, for example, information on movement (e.g., translation, curvature, and length) of the distal end 114 and the catheter 112 through the subject 110.

In conjunction, the model mapper 118 executing on the intraoperative imaging system 102 may obtain, identify, or otherwise access the model representation 132 (sometimes generally referred herein as a first tomogram) from the database 128. In some embodiments, the model mapper 118 may identify the model representation 132 based on an identifier for the subject 110 common with identifier for the subject 110 associated with the data 136 acquired via the endoscopic device 106. The model representation 132 may be derived from scanning of the volume 230 within the subject 110 prior to the invasive procedure. The model representation 132 may be a tomogram acquired from the tomograph 104 another tomographic imaging device. For example, the tomograph 104 may be an X-ray machine and the tomographic imaging device from which the model representation 132 is obtain may be a computed axial tomography (CAT) scanner.

The model representation 132 may be two-dimensional or three-dimensional, and may delineate or otherwise define an outline of the organ 130 within the scanning volume 230 of the subject 110. The definition may be in terms of coordinates or regions within the model representation 132. The model representation 132 may include or identify a representation of the organ 130, one or more cavities 405 (e.g., tracts for a lung) within the organ 130, and at least one target 410. The target 410 may be a region of interest (ROI) in or on the organ 130 of the subject 110, and may be, for example, a nodule, a lesion, a hemorrhage, or a tumor, among others, in or on the organ 130. In some embodiments, the target 410 may be manually identified within the model representation 132. For example, a clinician examining the model representation 132 may mark or annotate the target 410 using a graphical user interface before the invasive procedure on the subject 110. In some embodiments, the target 410 may be automatically detected in the model representation 132 using one or more computer vision techniques. For example, an object recognition algorithm (e.g., deep learning model, a scale-invariant feature transform (SIFT), or affine invariant feature detection) may be applied to the model representation 132 to identify one or more features corresponding to the target 410. Upon identification, the target 410 may be labeled in the model representation 132.

Using the data 136 from the endoscopic device 106, the model mapper 118 may determine or identify an estimated relative location 415A (sometimes herein generally referred to as a first relative location) of the distal end 114 of the endoscope 108 in relation to the target 410 in the model representation 132. The estimated relative location 415A may correspond to a displacement (defining a distance and angle) between the distal end 114 and the target 410. The estimated relative location 415A may differ from an actual relative location of the distal end 114 of the endoscopic device 106 physically in relation to the target 410 within the organ 130 of the subject 110. This may be because the estimated relative location 415A may be determined in terms of the model representation 132 derived from a scanning from prior to the invasive procedure. The features as defined in the model representation 132 may differ from the actual locations in the physical organ 130 of the subject 110.

In identifying, the model mapper 118 may identify or determine a point (e.g., a centroid defined in terms of (x, y, z)) for the distal end 114 within the model representation 132 based on the data 136 acquired via the endoscopic device 106. For example, the model mapper 118 may use the operational data from the endoscopic device 106 to estimate the point location of the distal end 114 within the cavity 405 of the organ 130. In addition, the model mapper 118 may identify a region (e.g., a volumetric region defined in terms of ranges of (x, y, z)) corresponding to the target 410 within the model representation 132. With the identifications, the model mapper 118 may calculate or determine the relative estimated location 415A based on a distance and angle between the point and the region. In some embodiments, the model mapper 118 may convert the distance and angle from pixel coordinates in the model representation 132 to a unit of measurement (e.g., millimeters, centimeters, or inches).

With the identification, the UI provider 124 (not shown) executing on the intraoperative imaging system 102 may provide the relative estimated location 415A in the model representation 132 for display. In providing, the UI provider 124 may present the model representation 132 and the relative estimated location 415A with the model representation 132 via the user interface 138. The user interface 138 may be used to provide a presentation or rendering of the model representation 132 from various aspects, such as a sagittal, coronal, axial, or transverse view, among others. In addition, the UI provider 124 may provide a visual representation of the endoscopic device 106 on the model representation 132 (e.g., as an overlay) via the user interface 138. The visual representation may correspond to at least a portion of the catheter 112 and the distal end 116 on the endoscopic device 106. The UI provider 124 may also provide a visual representation corresponding to the target 410 on the model representation 132 (e.g., as an overlay) via the user interface 138. In some embodiments, the UI provider 124 may generate an indicator identifying the estimated location 415A (e.g., as an overlay) on the model presentation 132 for presentation via the user interface 138. The indicator may be, for example, an arrow between the distal end 114 and the target 410 (e.g., as depicted) or the number in terms of unit of measurement for the relative estimated location 415A.

Referring now to FIG. 4B, depicted is a block diagram of a tomogram acquisition operation 430 for the system 100 for intraoperative medical imaging. As depicted, the tomogram processor 120 executing on the intraoperative imaging system 102 may retrieve, identify, or otherwise receive the tomogram 134 (sometimes generally referred to as the second tomogram) using the tomograph 104. The tomogram 134 may be acquired via the tomograph 104 in response to an activation. The tomogram 134 may be of the scanning volume 230 within the subject 110 at a time instance during the invasive procedure. In some embodiments, multiple tomograms 134 may be received from the tomograph 104 to obtain a more accurate depiction of the scanning volume 230 including the organ 130. In some embodiments, the time instance may correspond to or substantially correspond (e.g., less than 1 minute) a time of acquisition by the endoscopic device 106. In some embodiments, the time instance for acquisition of the tomogram 134 by the tomograph 104 may be within a time window (e.g., less than a 1 minute) of the time instance corresponding to the acquisition by the endoscopic device 106. For example, upon viewing the location of the distal end 114 in the model representation 132 rendered on the user interface 138, a clinician administering the invasive procedure may initiate the scanning of the scanning volume 320 using the tomograph 104.

The tomogram 134 may be two-dimensional or three-dimensional, and may identify or include the endoscopic device 106 (e.g., at least a portion of the catheter 112 and the distal end 114, the organ 130, cavities 405′ in the organ 130, and a feature 435 within the organ 130. As the tomogram 134 is acquired during the invasive procedure on the subject 110, the general shape of the organ 130 and the cavities 405′ may have shifted or be different from the outline of the organ 130 and cavities 405 as identified in the model representation 132. This may be because the tomogram 134 is acquired from the scanning volume 230 in the subject 110 closer to real-time during the invasive procedure, whereas the model representation 132 was acquired prior to the invasive procedure. In some embodiments, the tomogram 134 may delineate or otherwise define an outline of the organ 130 within the scanning volume 230 of the subject 110 in two or three-dimensions. When three-dimensional, the tomogram 134 may include a set of two-dimensional slices of the scanning volume 240 in the subject 110. In some embodiments, the tomogram 134 may be of the same imaging modality as the model representation 132. In some embodiments, the tomogram 134 may be of an imaging modality different from that of the model representation 132. For instance, the imaging modality for the tomogram 134 may be a X-ray imaging and the image modality for the model representation 132 may be a CT scan imaging.

The tomogram processor 120 may apply one or more computer vision techniques to the tomogram 134 to identify various objects from the tomogram 134. Using edge detection, the tomogram processor 120 may identify the organ 130 and one or more cavities 405′ within the tomogram 134. The edge detection applied by the tomogram processor 120 may include, for example, canny edge detector, Sobel operator, or differential operator, among others. Using feature detection, the tomogram processor 120 may identify or detect one or more features, such as the distal end 114, the catheter 112, or a region of interest (ROI) 435 (sometimes also referred herein as a target) in or on the organ 130 of the subject 110, among others. The ROI 435 may correspond to a nodule, a lesion, a hemorrhage, or a tumor, among others, in or on the organ 130, and may be the same type of feature as marked as the target 410 in the model representation 132. The feature detection applied by the tomogram processor 120 may include, for example, deep learning model, a scale-invariant feature transform (SIFT), or affine invariant feature detection, among others. In some embodiments, the tomogram processor 120 may label and store the identification of the organ 130, the cavities 405′, and the ROI 435 on the tomogram 134.

Referring now to FIG. 4C, depicted is a block diagram of an image registration operation 450 for the system for intraoperative medical imaging. As depicted, the registration handler 122 executing on the intraoperative imaging system 102 may register or perform an image registration between the tomogram 134 and the model representation 132. As discussed above, the model representation 132 may be acquired prior to the invasive procedure and the tomogram 134 may be during the invasive procedure. The image registration may be performed in accordance with any number of techniques. For example as discussed above, the registration handler 122 may perform a feature-based, multi-modal co-registration, among others, on the model representation 132 and the tomogram 134.

In performing the image registration, the registration handler 122 may identify the features (sometimes referred herein as is landmarks or markers) in the model representation 132 and the tomogram 134. The features may include, for example, the endoscopic device 106 (including at least a portion of the catheter 112 and the distal end 114), the organ 130, the cavity 405, and the target 410 detected by the model mapper 118 in the model representation 132. The features may also include, for example, the endoscopic device 106 (including at least a portion of the catheter 112 and the distal end 114), the organ 130, the cavity 405′, and the ROI 435 detected by the tomogram processor 120 in the tomogram 134. In some embodiments, the image registration may include the detection of the features in the model representation 132 by the model mapper 118 and in the tomogram 134 by the tomogram processor 120. The location and orientation of the features detected from the model representation 132 and those from the tomogram 134 may differ, as the model representation 132 was acquired prior to the invasive procedure while the tomogram 134 is acquired during.

With the detection, the registration handler 122 may compare the model representation 132 and the tomogram 134 to determine a correspondence between the features. The correspondence may indicate that the feature in the model representation 132 is the same type of object as the feature in the tomogram 134. In comparing, the registration handler 122 may align, match, or otherwise correlate the features detected from the model representation 132 and the corresponding features detected from the tomogram 134. To determine the correlation, the registration handler 122 may calculate or determine a degree of similarity between the feature in the model representation 132 to the feature in the tomogram 134. The degree of similarity may be based on properties (e.g., size, shape, color, and location) of the feature in the model representation 132 versus the properties of the feature in the tomogram 134. For example, both the ROI 435 and the target 410 may be associated with a tumorous growth within the lung, and thus may have higher similarity given the shape and size.

Upon determination, the registration handler 122 may compare the degree of similarity to a threshold. The threshold may delineate a value for the degree of similarity at which to determine that the feature in the model representation 132 matches the features in the tomogram 134. When the degree of similarity is determined to satisfy (e.g., greater than) the threshold, the registration handler 122 may determine that the features match or correspond. In this example, the registration handler 122 may determine that the ROI 435 detected from the tomogram 134 matches the target 410 identified by the model representation 132 as depicted, when the degree of similarity is high enough. In addition, registration handler 122 may determine that the distal end 114 as identified using the model representation 132 matches the distal end 114 detected in the tomogram 134. On the other hand, when the degree of similarity is determined to not satisfy (e.g., less than or equal to) the threshold, the registration handler 122 may determine that the features do not match or not correspond. The registration handler 122 may run the comparison to each combination of features identified in the model representation 132 and the tomogram 134.

Using the correspondences, the registration handler 122 may determine a set of transformation parameters for each matching feature common to the tomogram 134 and the model representation 132. The set of transformation parameters may define or identify differences in the visual representations of each feature between the tomogram 134 and the model representation 132. The set of transformation parameters may define or identify, for example, translation, rotation, reflection, scaling, or shearing from the feature in the model representation 132 to the feature in the tomogram 134, or vice-versa. For example, the distal end 114 as identified in the model representation 132 and the distal end 114 as detected from the tomogram 134 may have a difference in translation. Furthermore, the target 410 identified in the model representation 132 the ROI 435 detected from the tomogram 134 may have difference in scaling and shearing, among others.

From performing the image registration, the registration handler 122 may calculate or determine an actual relative location 415B (sometimes herein generally referred to as a first relative location) of the distal end 114 of the endoscope 108 in relation to the target 410 in the tomogram 134. The estimated relative location 415B may correspond to a displacement (defining a distance and angle) between the distal end 114 and the ROI 435. In some embodiments, the registration handler 122 may identify the feature corresponding to the distal end 114 and the feature corresponding to the ROI 435 in the tomogram 134. With the identifications, the registration handler 122 may identify or determine a point (e.g., a centroid defined in terms of (x, y, z)) for the distal end 114 within the tomogram 134. In addition, the model mapper 118 may identify a region (e.g., a volumetric region defined in terms of ranges of (x, y, z)) corresponding to the target 410 within the model representation 132. Based on these identifications, the registration handler 122 may calculate or determine a distance and angle between the point and the region. The registration handler 122 may also calculate or determine the actual relative location 415B based on the distance and angle. In some embodiments, the registration handler 122 may convert the distance and angle from pixel coordinates in the tomogram 134 to a unit of measurement (e.g., millimeters, centimeters, or inches).

In addition, the registration handler 122 may calculate or determine one or more deviation measures between the feature in the model representation 132 and the corresponding feature in the tomogram 134. The determination of the deviation measure may be based on the set of transform parameters determined from the image registration. The deviation measure may identify or include, for example, at least one deviation 455 corresponding to a displacement between the feature in the tomogram 134 and the feature in the model representation 132. The deviation 455 may identify or include the displacement between the feature in the tomogram 134 and the feature in the model representation 132. In some embodiments, the deviation measure may also include one or more of the set of transform parameters between the target 410 in the model representation 132 and the ROI 435 in the tomogram 134. For example, the deviation measure may include a difference in size, position, or orientation, among others, between the target 410 in the model representation 132 and the ROI 435 in the tomogram 134.

With the determinations, the UI provider 124 may provide various data in connection with the image registration between the model representation 132 and the tomogram 134 for display via the user interface 138 on the display 108. The presentation of the user interface 138 may be during the invasive procedure. The user interface 138 may be a graphical user interface for rendering, displaying, or otherwise presenting data derived from the model representation 132, the tomogram 134, and the image registration. The user interface 138 may be used to provide a presentation or rendering of the tomogram 134 from various aspects, such as a sagittal, coronal, axial, or transverse view, among others. In some embodiments, the user interface 138 may present the model representation 132 (including representations of the endoscopic device 106, the organ 130, the cavity 405, the target 410). In some embodiments, the user interface 138 may present the estimated relative location 415A or the actual relative location 415B on the model representation 132. In some embodiments, the user interface 138 may include a location of the target 410 within the model representation 132. In some embodiments, the user interface 138 may include the tomogram 134 (including representations of the endoscopic device 106, the organ 130, the cavity 405′, and the ROI 435). In some embodiments, the user interface 138 may include the actual relative location 415B and the deviation measures on the tomogram 134. In some embodiments, the user interface 138 may include the location of the ROI 435 in the tomogram 134.

At a subsequent time instance during the invasive procedure on the subject 110, the operations and functionalities of the intraoperative imaging system 102 may be repeated. For example, the clinician viewing the information on the user interface 138 may make adjustments (e.g., rotation or movement) to the positioning of the distal end 114 of the endoscopic device 106 within the subject 110. The endoscopic interface 116 may continue to receive the data 136 from the endoscopic device 106. The model mapper 118 may update the relative estimated location 415A based on the new data 136. The tomogram processor 120 may receive another tomogram 134 from the tomograph 104 upon activation by the clinician. The tomogram processor 120 may apply computer vision techniques to detect the features within the tomogram 134. The registration handler 122 may perform another image registration on the new tomogram 134 and the model representation 132. With the image registration, the registration handler 122 may determine various information as discussed above (e.g., the actual relative location 415B and deviation 455). The UI provider 124 may update the information displayed via the user interface 138. In this manner, the determining positioning of the endoscopic device 106 inserted within the subject 101 may be more accurate and precise, and may have a higher chance at successfully reaching the target 410 within the model representation 132.

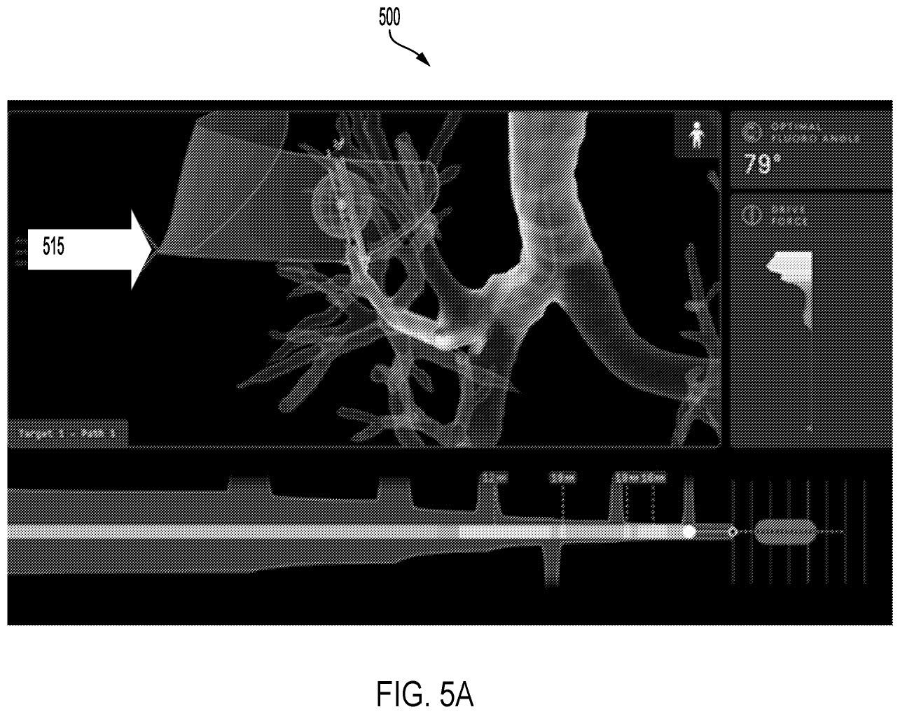

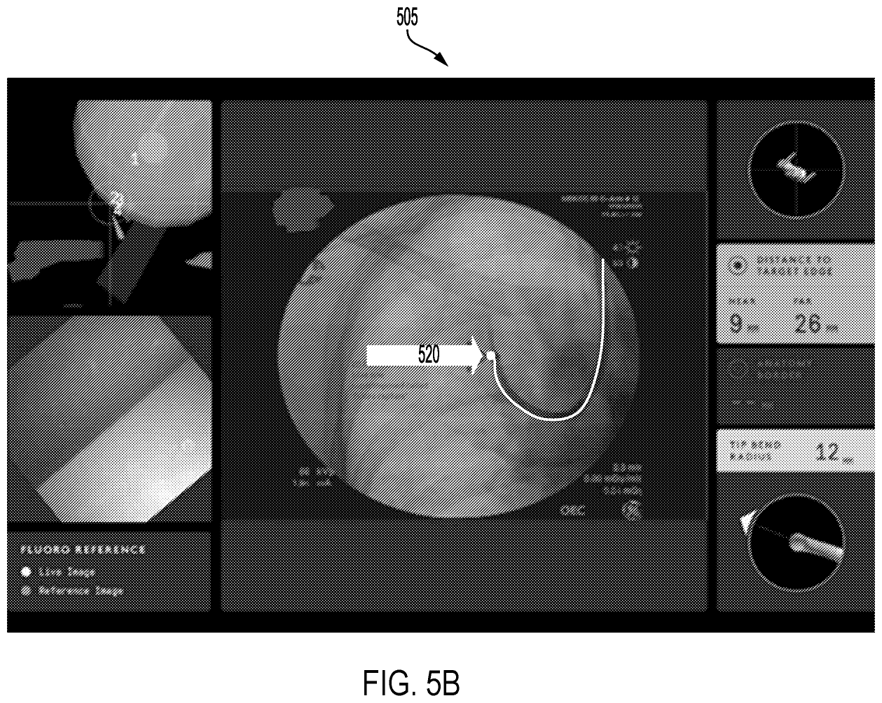

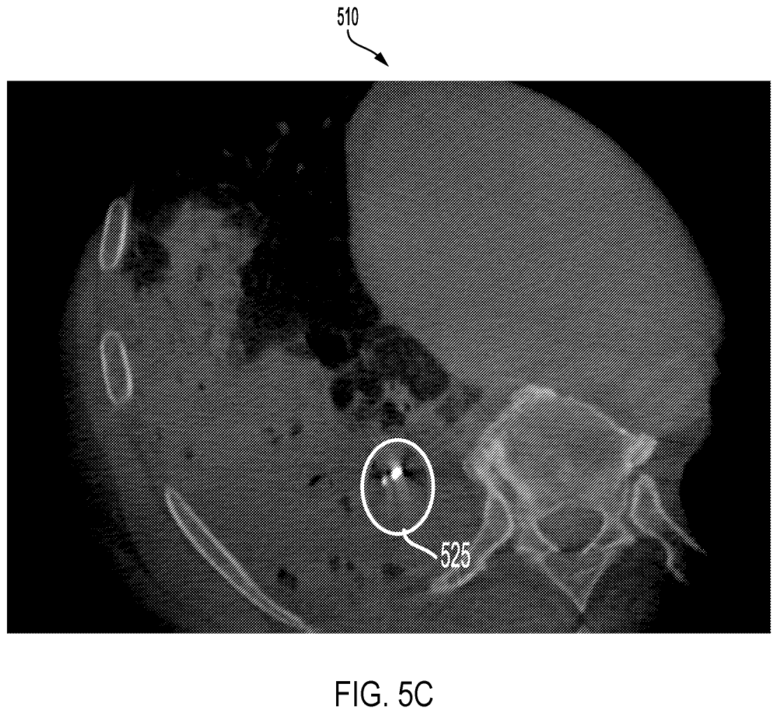

Referring now to FIGS. 5A-5C, depicted are screenshots 500-510 of graphical user interface provided by the system for intraoperative medical imaging. The screenshots 500-510 may be of the user interface 138. In screenshot 500, the user interface 138 may include a virtual 3D position map of the robotic bronchoscope within the lung using a prior CT scan (e.g., the representation model 132) and real-time endoscopic visual landmarks. The user interface 138 may include estimate of a virtual distances between the distal end 114 and visual landmarks. The user interface 138 may include at least one indicator 515 of an anatomical visual landmark. The virtual distance may include, for example: the distance between the robotic catheter to the proximal end of the target lesion, distance of the robotic catheter to the distal end of the lesion, and distance of the robotic catheter to an anatomical landmark to be avoided, among others. In screenshot 505, the user interface 138 may include a fluoroscope image of the endoscopic device 106 within the subject 110, with the distal end 114 marked with an indicator 520. In screenshot 510, the user interface 138 may include a tomogram 134 produced by the tomograph 104 with the distal end 114 of the endoscopic device 106 may appear in one region 525.

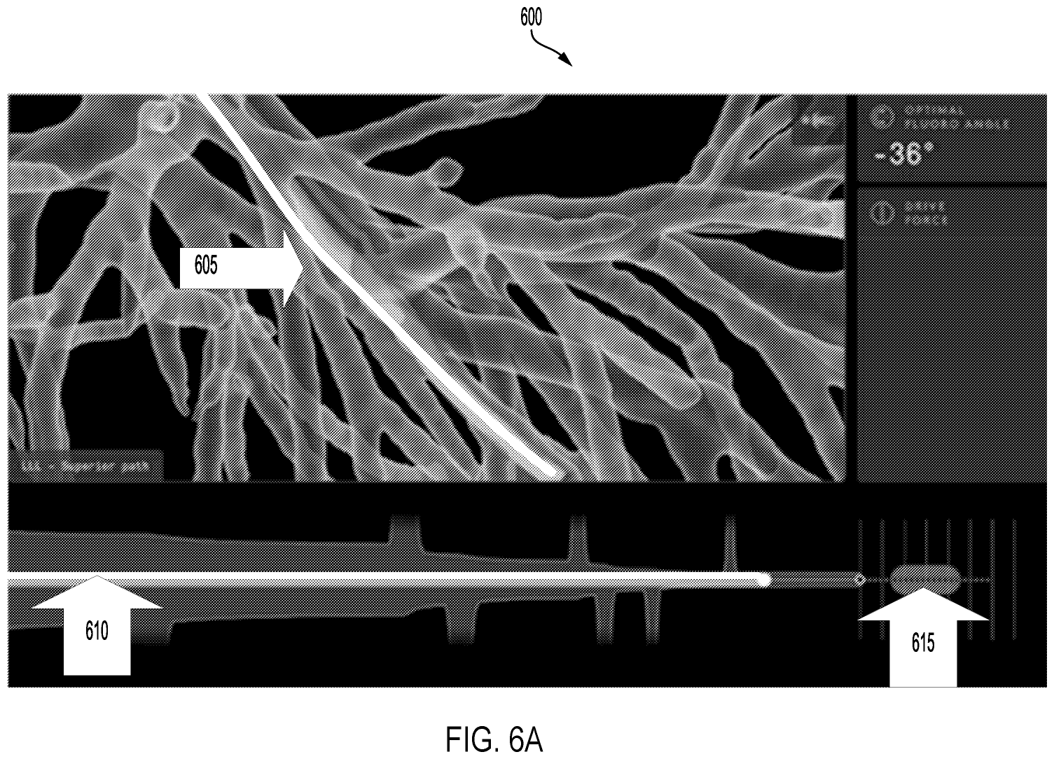

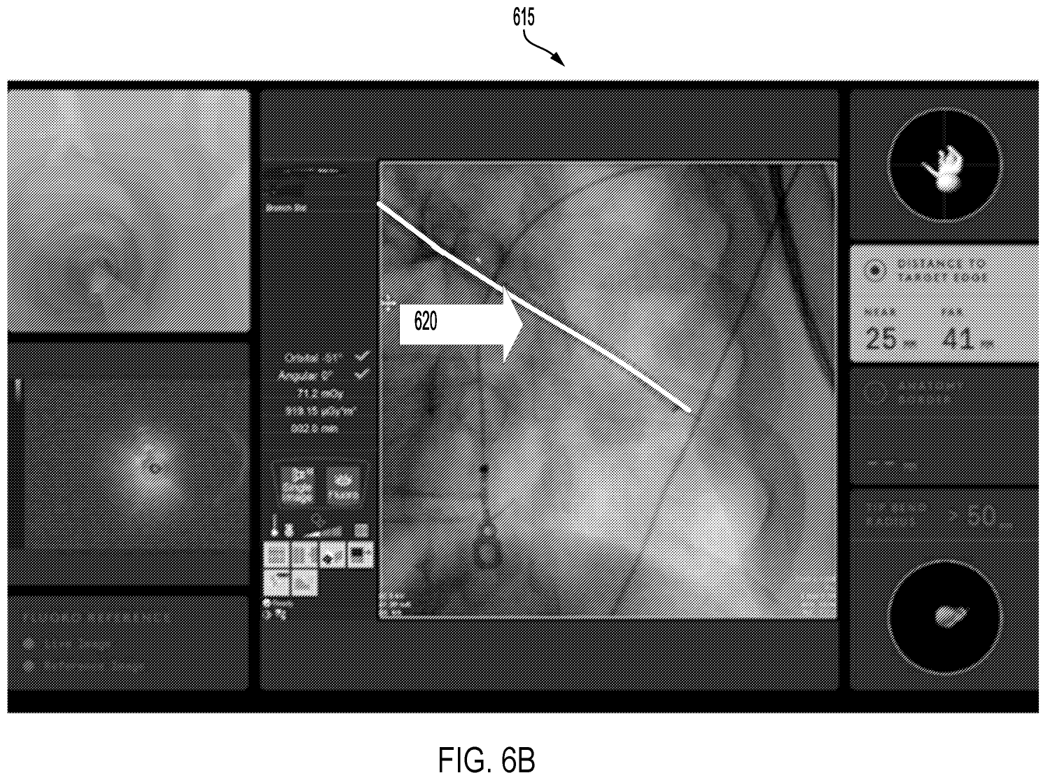

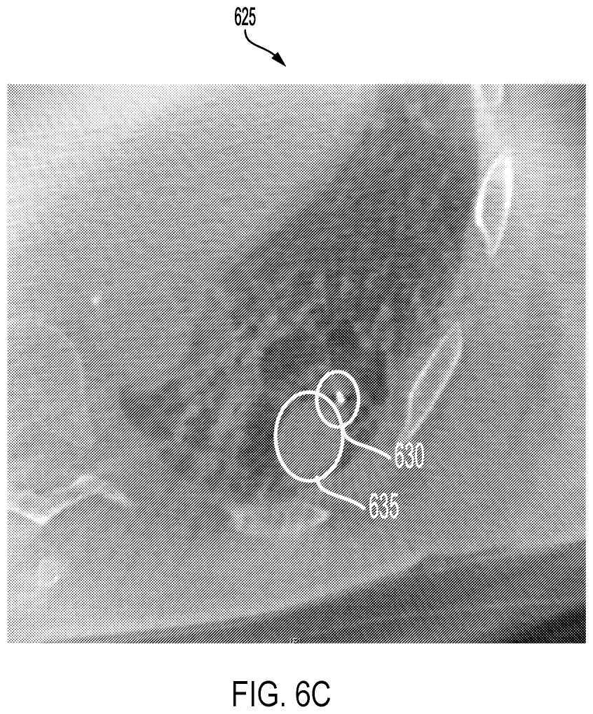

Referring now to FIGS. 6A-6C, depicted are screenshots of graphical user interface provided by the system for intraoperative medical imaging. The screenshots may be of the user interface 138. In screenshot 600, the user interface 138 may include a virtual 3D position map of the robotic bronchoscope within the lung using a prior CT scan and real-time endoscopic visual landmarks. The user interface 138 may include a highlight 605 corresponding to the catheter 112 within the three-dimensional representation of the lung of the subject 110. Along the bottom, the user interface 138 may include an indicator 610 for the catheter 112 and the distal end 114 through the lung of the subject 110 and an indicator 615 for an anatomical landmark to be avoided. In screenshot 615, the user interface 138 may include a fluoroscope image of the endoscopic device 106 within the subject 110, with the catheter 112 indicated with a highlight 620. In screenshot 625, the user interface 138 may include the tomogram 134 with an indicator 630 for the distal end 114 and an indicator 635 for the ROI 435.

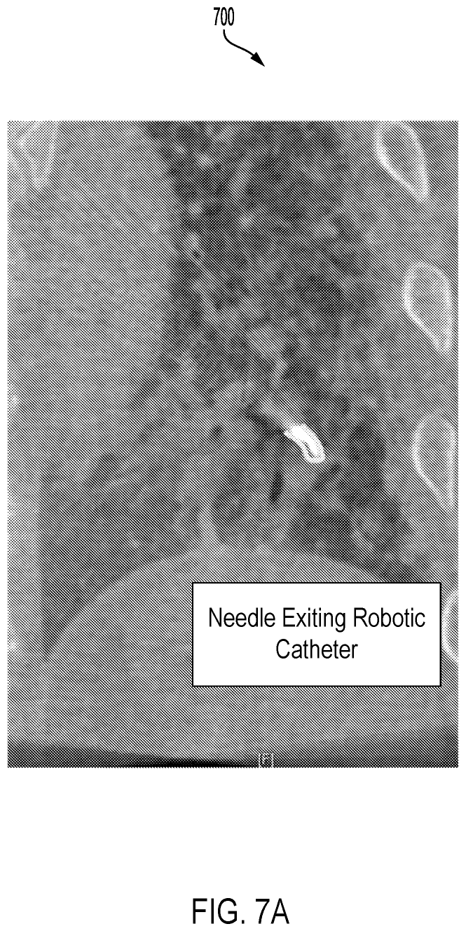

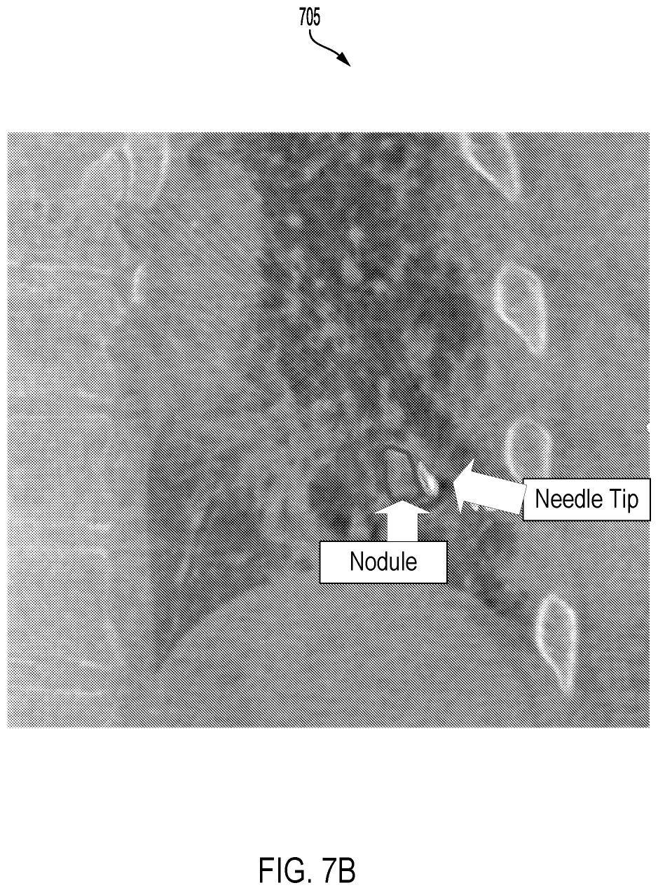

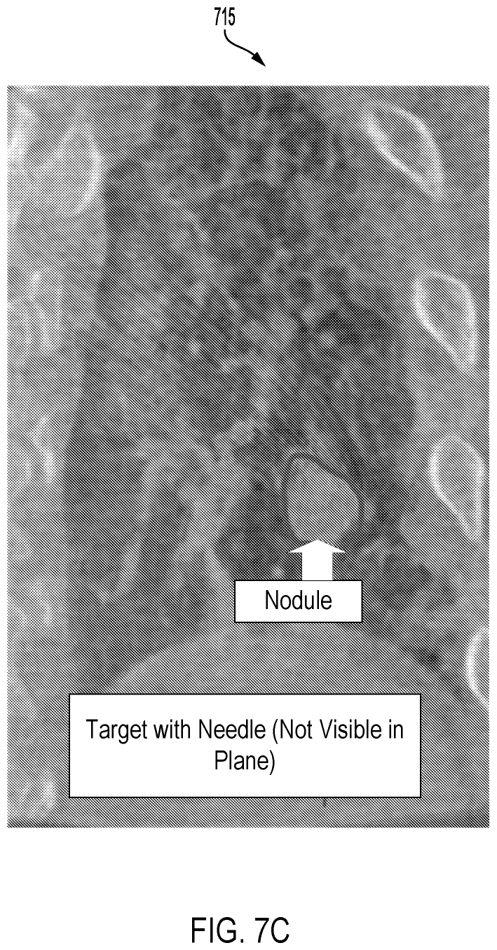

Referring now to FIGS. 7A-7C, depicted are screenshots 700-710 of graphical user interface provided by the system for intraoperative medical imaging at various time instances during the invasive procedure. In screenshot 700, the user interface 138 may present the tomogram 134 in which a needle (e.g., on the distal end 114) is exiting the catheter 112 of the endoscopic device 106 at a first time instance. In screenshot 705, the user interface 138 may present the needle tip approaching the nodule (e.g., ROI 435) in the tomogram 134, as the operator causes the endoscopic device 108 to move toward the nodule. In screenshot 710, the user interface 138 may present the needle within the nodule, thus rendering the needle invisible in the plane of the tomogram 134. s

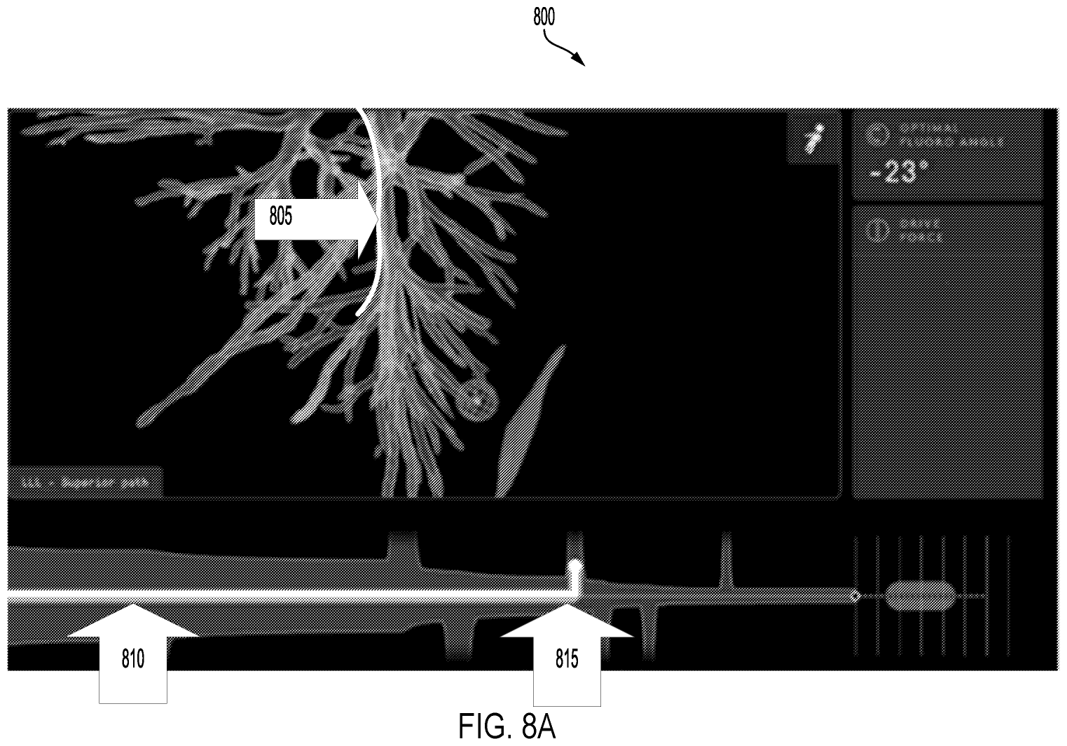

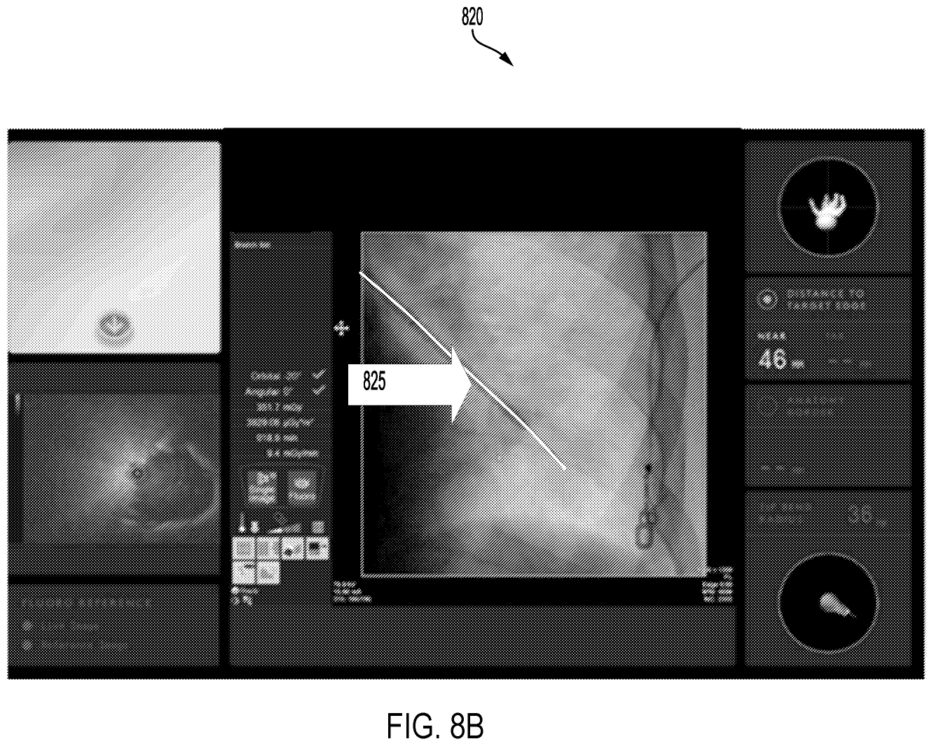

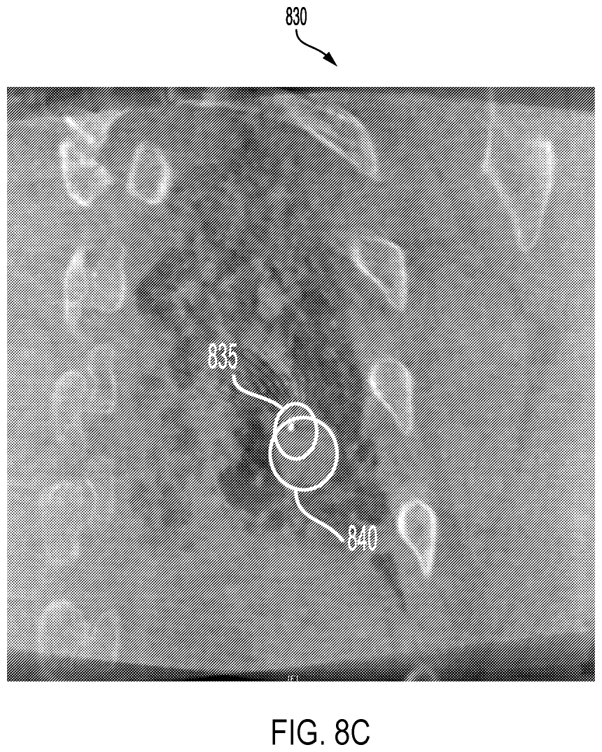

Referring now to FIGS. 8A-8C, depicted are screenshots of graphical user interface provided by the system for intraoperative medical imaging. The screenshots may be of the user interface 138. In screenshot 800, the user interface 138 may include a virtual 3D position map of the robotic bronchoscope within the lung using a prior CT scan and real-time endoscopic visual landmarks. The user interface 138 may include a highlight 805 corresponding to the catheter 112 within the three-dimensional representation of the lung of the subject 110. Along the bottom, the user interface 138 may include an indicator 810 for the catheter 112 and the distal end 114 through the lung of the subject 110 and an maker 815 showing a bending of the catheter 112. In screenshot 820, the user interface 138 may include a fluoroscope image of the endoscopic device 106 within the subject 110, with the catheter 112 indicated with a highlight 825. In screenshot 830, the user interface 138 may include the tomogram 134 with an indicator 835 for the distal end 114 and an indicator 840 for the ROI 435.

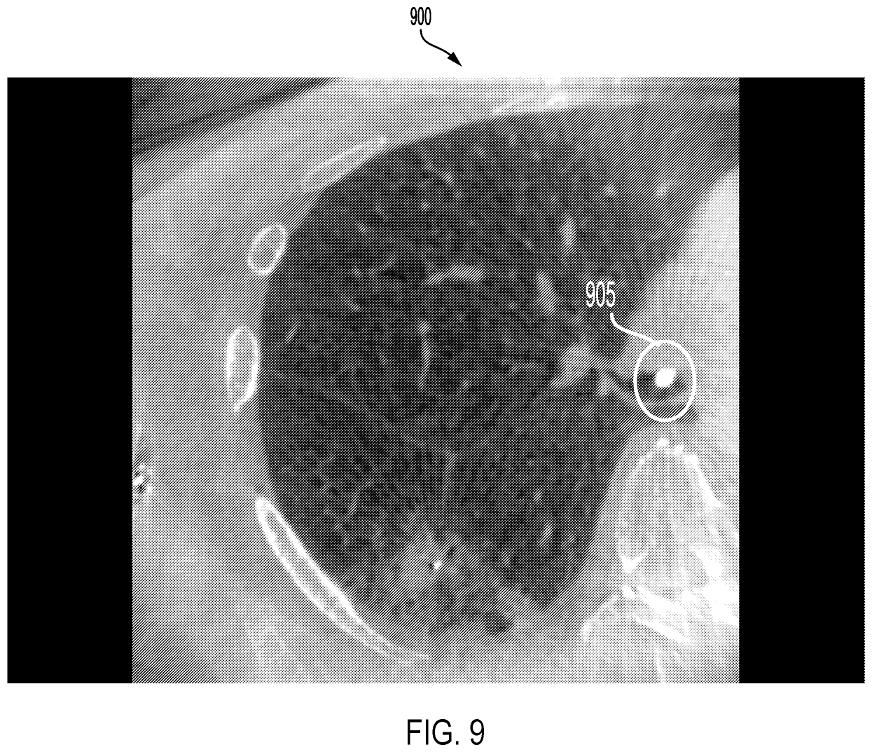

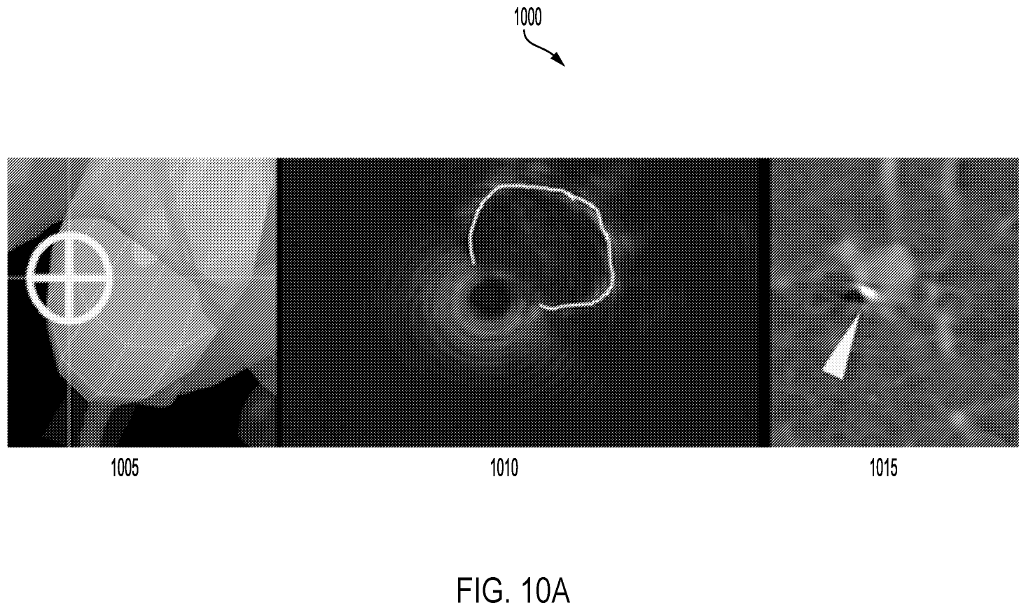

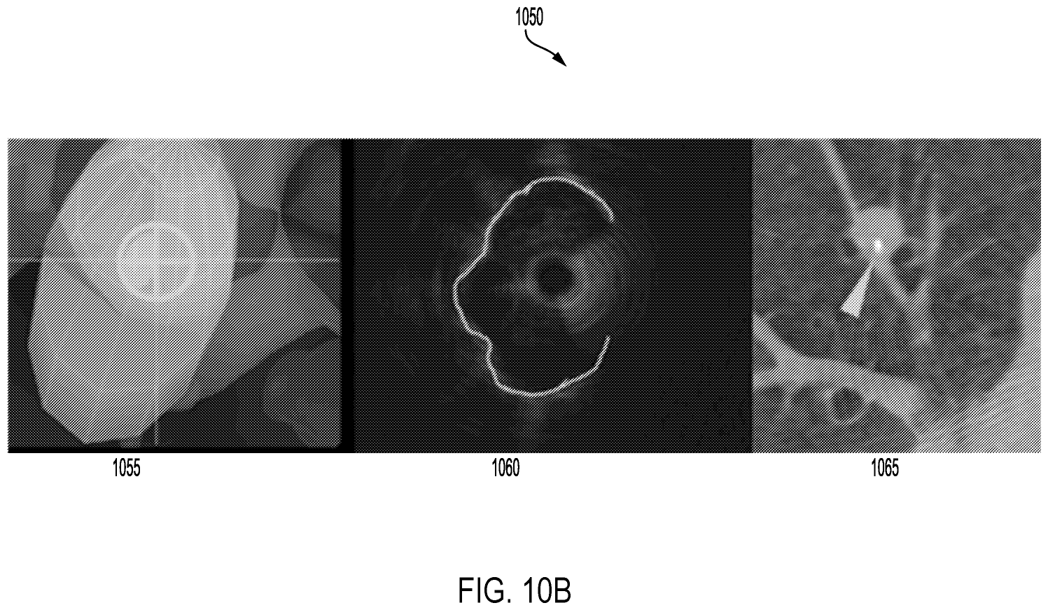

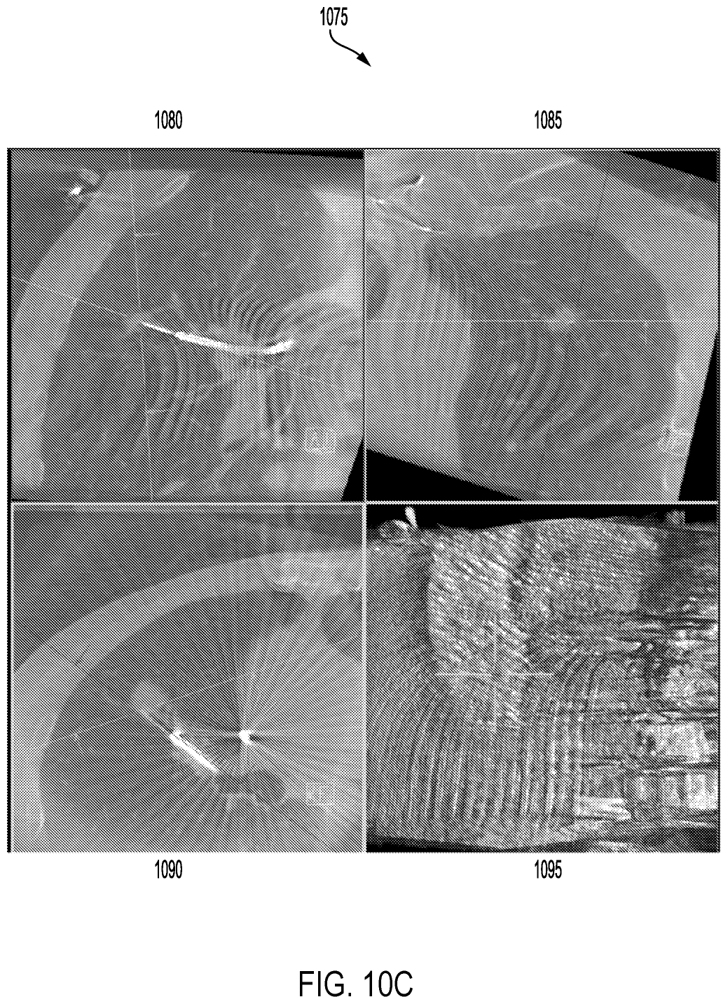

Referring now to FIG. 9, depicted is a screenshot of a graphical user interface provided by the system for intraoperative medical imaging As depicted in screenshot 900, the user interface 138 may include an indicator 905 corresponding to the distal end 114 (e.g., a needle) of the endoscopic device 106. Referring now to FIGS. 10A-10C, depicted are screenshots of graphical user interface provided by the system for intraoperative medical imaging. In screenshot 1000, the user interface 138 may include: an image 1005 for the model representation 132 acquired before the procedure showing an object corresponding to a positioning of the endoscopic device 106, an image 1010 for the ultrasound data acquired via the endoscopic device 106, and an image 1015 for the tomogram 134 acquired during the procedure. In screenshot 1050, the user interface 138 may include: an image 1055 for the model representation 132 acquired before the procedure showing an object corresponding to a positioning of the endoscopic device 106, an image 1060 for the ultrasound data acquired via the endoscopic device 106, and an image 1065 for the tomogram 134 acquired during the procedure. In screenshot 1075, the user interface 138 may include: an image 1080 of the tomogram 134 from a sagittal axis, an image 1085 of the tomogram 134 from a coronal axis, an image 1090 of the tomogram 134 from an axial perspective, and an image 1095 of the tomogram 134 in a three-dimensional perspective.

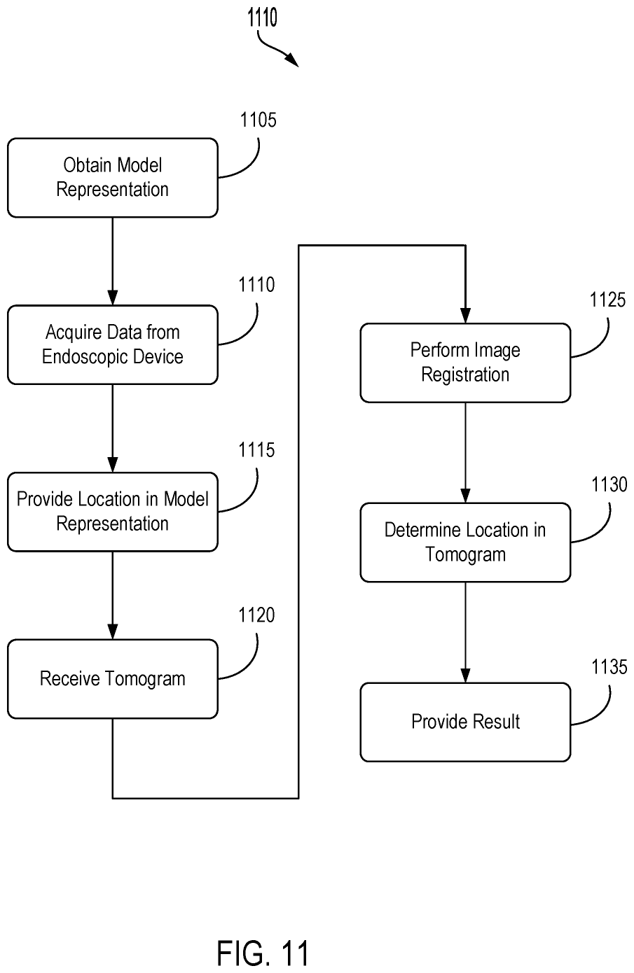

Referring now to FIG. 11, depicted is a flow diagram of a method 500 of intraoperative medical imaging. The method 1100 can be performed or implemented using any of the components detailed herein in conjunction with FIGS. 1-4C or FIG. 12. In the method 1100, a computing system may obtain a model representation (1105). The computing system may acquire data from an endoscopic device (1110). The computing system may provide a location in the model representation (1115). The computing system may receive a tomogram (1120). The computing system may perform image registration (1125). The computing system may determine a location in tomogram (1130). The computing system may provide a result (1135).

### B. Computing and Network Environment

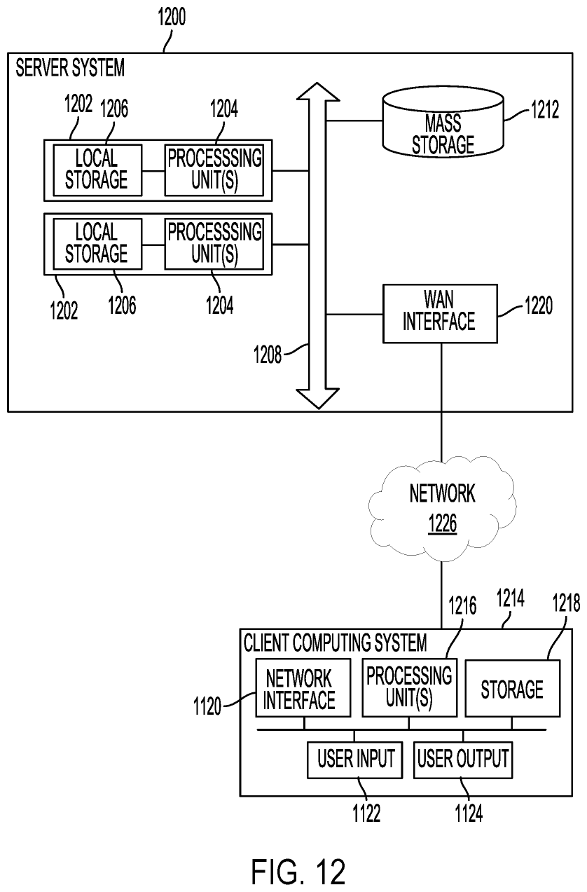

Various operations described herein can be implemented on computer systems. FIG. 12 shows a simplified block diagram of a representative server system 1200, client computer system 1214, and network 1226 usable to implement certain embodiments of the present disclosure. In various embodiments, server system 1200 or similar systems can implement services or servers described herein or portions thereof. Client computer system 1214 or similar systems can implement clients described herein. The system 100 described herein can be similar to the server system 1200. Server system 1200 can have a modular design that incorporates a number of modules 1202 (e.g., blades in a blade server embodiment); while two modules 1202 are shown, any number can be provided. Each module 1202 can include processing unit(s) 1204 and local storage 1206.

Processing unit(s) 1204 can include a single processor, which can have one or more cores, or multiple processors. In some embodiments, processing unit(s) 1204 can include a general-purpose primary processor as well as one or more special-purpose co-processors such as graphics processors, digital signal processors, or the like. In some embodiments, some or all processing units 1204 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1204 can execute instructions stored in local storage 1206. Any type of processors in any combination can be included in processing unit(s) 1204.

Local storage 1206 can include volatile storage media (e.g., DRAM, SRAM, SDRAM, or the like) and/or non-volatile storage media (e.g., magnetic or optical disk, flash memory, or the like). Storage media incorporated in local storage 1206 can be fixed, removable or upgradeable as desired. Local storage 1206 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1204 need at runtime. The ROM can store static data and instructions that are needed by processing unit(s) 1204. The permanent storage device can be a non-volatile read-and-write memory device that can store instructions and data even when module 1202 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1206 can store one or more software programs to be executed by processing unit(s) 1204, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 1204 cause server system 1200 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1204. Software can be implemented as a single program or a collection of separate programs or program modules that interact as desired. From local storage 1206 (or non-local storage described below), processing unit(s) 1204 can retrieve program instructions to execute and data to process in order to execute various operations described above.

In some server systems 1200, multiple modules 1202 can be interconnected via a bus or other interconnect 1208, forming a local area network that supports communication between modules 1202 and other components of server system 1200. Interconnect 1208 can be implemented using various technologies including server racks, hubs, routers, etc.

A wide area network (WAN) interface 1210 can provide data communication capability between the local area network (interconnect 1208) and the network 1226, such as the Internet. Technologies can be used, including wired (e.g., Ethernet, IEEE 802.3 standards) and/or wireless technologies (e.g., Wi-Fi, IEEE 802.11 standards).

In some embodiments, local storage 1206 is intended to provide working memory for processing unit(s) 1204, providing fast access to programs and/or data to be processed while reducing traffic on interconnect 1208. Storage for larger quantities of data can be provided on the local area network by one or more mass storage subsystems 1212 that can be connected to interconnect 1208. Mass storage subsystem 1212 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1212. In some embodiments, additional data storage resources may be accessible via WAN interface 1210 (potentially with increased latency).

Server system 1200 can operate in response to requests received via WAN interface 1210. For example, one of modules 1202 can implement a supervisory function and assign discrete tasks to other modules 1202 in response to received requests. Work allocation techniques can be used. As requests are processed, results can be returned to the requester via WAN interface 1210. Such operation can generally be automated. Further, in some embodiments, WAN interface 1210 can connect multiple server systems 1200 to each other, providing scalable systems capable of managing high volumes of activity. Other techniques for managing server systems and server farms (collections of server systems that cooperate) can be used, including dynamic resource allocation and reallocation.

Server system 1200 can interact with various user-owned or user-operated devices via a wide-area network such as the Internet. An example of a user-operated device is shown in FIG. 12 as client computing system 1214. Client computing system 1214 can be implemented, for example, as a consumer device such as a smartphone, other mobile phone, tablet computer, wearable computing device (e.g., smart watch, eyeglasses), desktop computer, laptop computer, and so on.

For example, client computing system 1214 can communicate via WAN interface 1210. Client computing system 1214 can include computer components such as processing unit(s) 1216, storage device 1218, network interface 1220, user input device 1222, and user output device 1224. Client computing system 1214 can be a computing device implemented in a variety of form factors, such as a desktop computer, laptop computer, tablet computer, smartphone, other mobile computing device, wearable computing device, or the like.

Processor 1216 and storage device 1218 can be similar to processing unit(s) 1204 and local storage 1206 described above. Suitable devices can be selected based on the demands to be placed on client computing system 1214; for example, client computing system 1214 can be implemented as a “thin” client with limited processing capability or as a high-powered computing device. Client computing system 1214 can be provisioned with program code executable by processing unit(s) 1216 to enable various interactions with server system 1200.

Network interface 1220 can provide a connection to the network 1226, such as a wide area network (e.g., the Internet) to which WAN interface 1210 of server system 1200 is also connected. In various embodiments, network interface 1220 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 1222 can include any device (or devices) via which a user can provide signals to client computing system 1214; client computing system 1214 can interpret the signals as indicative of particular user requests or information. In various embodiments, user input device 1222 can include any or all of a keyboard, touch pad, touch screen, mouse or other pointing device, scroll wheel, click wheel, dial, button, switch, keypad, microphone, and so on.

User output device 1224 can include any device via which client computing system 1214 can provide information to a user. For example, user output device 1224 can include a display to display images generated by or delivered to client computing system 1214. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1224 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1204 and 1216 can provide various functionality for server system 1200 and client computing system 1214, including any of the functionality described herein as being performed by a server or client, or other functionality.

It will be appreciated that server system 1200 and client computing system 1214 are illustrative and that variations and modifications are possible. Computer systems used in connection with embodiments of the present disclosure can have other capabilities not specifically described here. Further, while server system 1200 and client computing system 1214 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. For instance, although specific examples of rules (including triggering conditions and/or resulting actions) and processes for generating suggested rules are described, other rules and processes can be implemented. Embodiments of the disclosure can be realized using a variety of computer systems and communication technologies including but not limited to specific examples described herein.

Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method for intraoperative medical imaging, comprising:
accessing, by a computing system from a database, a first tomogram derived from scanning a volume within a subject prior to an invasive procedure, the first tomogram identifying a target within the volume of the subject;
acquiring, by the computing system, data via an endoscopic device at least partially disposed within the subject at a time instance during the invasive procedure;
providing, by the computing system for display, in the first tomogram of the subject, a first relative location of a distal end of the endoscopic device and the target based on the data;
receiving, by the computing system using a tomograph, a second tomogram of the volume within the subject at the time instance during the invasive procedure, the second tomogram including the distal end of the endoscopic device;
registering, by the computing system, the second tomogram received from the tomograph during the invasive procedure with the first tomogram obtained prior to the invasive procedure to determine a second relative location of the distal end of the endoscopic device and the target within the subject; and
providing, by the computing system for display, the second relative location of the distal end and the target within the subject during the invasive procedure.

2. The method of claim 1, further comprising:
receiving, by the computing system using the tomograph, a third tomogram of the volume within the subject at a second time instance during the invasive procedure after the time instance, the third tomogram including the distal end of the endoscope moved subsequent to provision of the second relative location;
registering, by the computing system, the third tomogram received from the tomograph at the second time instance with the first tomogram received prior to the invasive procedure to determine a third relative location of the distal end of the endoscopic device and the target within the subject; and
providing, by the computing system for display, the third relative location of the distal end and the target within the subject.

3. The method of claim 1, further comprising providing, by the computing system, a graphical user interface for display of one or more of: the first tomogram, the first relative location or the second relative location of the distal end in the first tomogram, a first location of the target in the first tomogram, the second tomogram, the second relative location of the distal end in the second tomogram, and a second location of the target in the second tomogram.

4. The method of claim 1, wherein accessing the first tomogram further comprises identifying a three-dimensional representative model derived from scanning the volume within the subject prior to the invasive procedure, the three-dimensional representative model identifying an organ within the subject, one or more cavities within the organ, and the target.

5. The method of claim 1, wherein acquiring the data further comprises acquiring, via the endoscopic device, the data comprising at least one of image data acquired via the distal end of the endoscopic device and operational data identifying a translation of the endoscope through the subject.

6. The method of claim 1, wherein receiving the second tomogram further comprises receiving, using the tomograph, the second tomogram in at least one of a two-dimensional space or a three-dimensional space, the second tomogram in an imaging modality different from an imaging modality of the first tomogram.

7. The method of claim 1, wherein registering the second tomogram with the first tomogram further comprises determining a displacement between the distal end of the endoscopic device and the target within the subject.

8. The method of claim 1, wherein registering the second tomogram with the first tomogram further comprises determining a displacement between the target in the first tomogram and the target in the second tomogram within the subject.

9. The method of claim 1, wherein registering the second tomogram with the first tomogram further comprises determining a difference in size between the target in the first tomogram and the target in the second tomogram within the subject.

10. The method of claim 1, wherein the invasive procedure further comprises a bronchoscopy, the distal end of the endoscopic device is inserted through a tract in a lung of the subject, and the volume of the subject scanned at least partially includes the lung.

11. A system for intraoperative medical imaging, comprising:
a computing system having one or more processors coupled with memory, configured to:
access, from a database, a first tomogram derived from scanning a volume within a subject prior to an invasive procedure, the first tomogram identifying a target within the volume of the subject;
acquire data via an endoscopic device at least partially disposed within the subject at a time instance during the invasive procedure;
provide, for display, in the first tomogram of the subject, a first relative location of a distal end of the endoscopic device and the target based on the data;
receive, using a tomograph, a second tomogram of the volume within the subject at the time instance during the invasive procedure, the second tomogram including the distal end of the endoscopic device;
register the second tomogram received from the tomograph during the invasive procedure with the first tomogram obtained prior to the invasive procedure to determine a second relative location of the distal end of the endoscopic device and the target within the subject; and
provide, for display, the second relative location of the distal end and the target within the subject during the invasive procedure.

12. The system of claim 11, wherein the computing system is further configured to:
receive, using the tomograph, a third tomogram of the volume within the subject at a second time instance during the invasive procedure after the time instance, the third tomogram including the distal end of the endoscope moved subsequent to provision of the second relative location;
register the third tomogram received from the tomograph at the second time instance with the first tomogram received prior to the invasive procedure to determine a third relative location of the distal end of the endoscopic device and the target within the subject; and
provide, for display, the third relative location of the distal end and the target within the subject.

13. The system of claim 11, wherein the computing system is further configured to provide a graphical user interface for display of one or more of: the first tomogram, the first relative location or the second relative location of the distal end in the first tomogram, a first location of the target in the first tomogram, the second tomogram, the second relative location of the distal end in the second tomogram, and a second location of the target in the second tomogram.

14. The system of claim 11, wherein the computing system is further configured to identify a three-dimensional representative model derived from scanning the volume within the subject prior to the invasive procedure, the three-dimensional representative model identifying an organ within the subject, one or more cavities within the organ, and the target.

15. The system of claim 11, wherein the computing system is further configured to acquire, via the endoscopic device, the data comprising at least one of image data acquired via the distal end of the endoscopic device and operational data identifying a translation of the endoscope through the subject.

16. The system of claim 11, wherein the computing system is further configured to receive, using the tomograph, the second tomogram in at least one of a two-dimensional space or a three-dimensional space, the second tomogram in an imaging modality different from an imaging modality of the first tomogram.

17. The system of claim 11, wherein the computing system is further configured to register the second tomogram with the first tomogram to determine a displacement between the distal end of the endoscopic device and the target within the subject.

18. The system of claim 11, wherein the computing system is further configured to register the second tomogram with the first tomogram to determine a displacement between the target in the first tomogram and the target in the second tomogram within the subject.

19. The system of claim 11, wherein the computing system is further configured to register the second tomogram with the first tomogram to determine a difference in size between the target in the first tomogram and the target in the second tomogram within the subject.

20. The system of claim 11, wherein the invasive procedure further comprises a bronchoscopy, the distal end of the endoscopic device is inserted through a tract in a lung of the subject, and the volume of the subject scanned at least partially includes the lung.
